(19) United States
(12) Patent Application Publication
(10) Pub. No.: US 2018/0280013 A1
(43) Pub. Date: Oct. 4, 2018

(54) SURGICAL SUPPORT DEVICE AND METHODS OF USE

(71) Applicant: BEACON SURGICAL INSTRUMENTS, LLC, Dobbs Ferry, NY (US)

(72) Inventors: Sundaram RAVIKUMAR, Briarcliff Manor, NY (US); Guy OSBORNE, Trumball, CT (US); Harry Allan ALWARD, Shelton, CT (US)

(73) Assignee: Beacon Surgical Instruments, LLC, Dobbs Ferry, NY (US)

(21) Appl. No.: 15/572,958

(22) PCT Filed: Jan. 22, 2016
(86) PCT No.: PCT/US2016/014558

§ 371 Date: Nov. 9, 2017

Related U.S. Application Data
(60) Provisional application No. 62/160,566, filed on May 12, 2015.
(60) Provisional application No. 62/107,316, filed on Jan. 23, 2015.

(51) Int. Cl. A61B 17/02; A61L 31/06

(52) U.S. Cl. CPC A61B 17/0218; A61B 2017/00526; A61L 31/06

(57) ABSTRACT

A surgical support device including a housing containing a supporting means and a band or sling which are deployed out of an aperture in the housing specimen via an activator or deployment mechanism. The deployed supporting means and band or sling are configured to be capable of retracting, supporting and maintaining a body organ or tissue during a surgical procedure.

### RELATED APPLICATIONS

This application claims priority from PCT Application Serial No. PCT/US2016/014,558 filed Jan. 22, 2016, provisional application Ser. No. 62/210,7316 filed Jan. 23, 2015, and provisional application Ser. No. 62/160,566 filed on May 12, 2015.

### FIELD OF THE INVENTION

The present invention relates to a surgical support device for use in surgery to retract, contain and support a body organ or tissue and methods of use of the inventive device.

### BACKGROUND OF THE INVENTION

During many surgical procedures it is necessary to mobilize and move certain organs. For instance, a portion of the small intestine, large intestine or the bowel may have to be moved during abdominal surgery while the surgeon addresses a different target organ. Other surgeries may require other organs to be moved so that surgery on a target organ or tissue may be achieved. The term “target organ or tissue” is used for example to mean the organ or tissue which is the subject of a surgical procedure, such as the uterus during a hysterectomy and the term “non-target organ or tissue” is used for example to mean surrounding organs and tissue which are not the subject of the same surgical procedures, for example a bowel or intestine during a hysterectomy.

During open surgical procedures, the surgeon can accomplish the movement of organs, including non-target organs, manually without significant risk of damaging the non-target organ, such as manually moving a bowel during a uterine procedure. However, during laparoscopic surgical procedures, manual movement of the non-target organ is no longer possible and the surgeon's view of the operative site is limited to what can be shown via the endoscope or other inner camera. Thus, the surgeon's view of the operating site is limited and non-target tissue and organs may obstruct the surgeon's view of the surgical site and location of the target tissue or organ. During laparoscopy, such as abdominal surgery, an incision is made in the abdomen sufficient for insertion of a 10 mm trocar (access devices) which forms pneumoperitoneum by putting gas (e.g. CO2 gas) into the abdomen during endoscopic surgery so as to create the space and visibility for the endoscopic surgery, and allows an endoscope and one or more surgical instruments to be inserted into the abdomen either through the same trocar or via additional incisions with or without trocars. Certain minimally invasive surgeries may use four or more trocars. The pneumoperitoneum must be maintained so as to provide an extended surgical site area for the surgeon to be able to access the target organ or tissue without his or her view being obstructed.

In order to achieve movement of the non-target organ during laparoscopic procedures, various clamps and graspers have been developed. For example, to move a non-target bowel during an abdominal or uterine surgery a Babcock clamp may be used, which fits through the trocar and grips directly onto the bowel. The surgeon will utilize the clamp for moving the bowel appropriately during operative procedures. Liver retractors are also known to be used during various surgical procedures. The surgeon may then insert various packing materials such as sponges and towels to support and hold the non-target organ out of the area where the surgery is taking place on the target organ or tissue. However, the risk of infection from the packing materials is present as well as the problem where a single sponge, gauze or towels is left within the body after being closed following the surgical procedure. Further, manipulation of cotton operating room towels within the abdominal cavity in abdominal surgery has been associated with the formation of peritoneal adhesions.

In various laparoscopic surgical procedures, it is desirable to position an internal organ so that it does not interfere with the surgical procedure. This is not only for the surgeon's convenience but also because of the desirability of avoiding damage to the organ. For example, in a typical gynecological operative laparoscopy, such as an oophorectomy (removal of the ovaries=target organ), it is very often necessary to manipulate or position the uterus as a non-target organ while maintaining the pneumoperitoneum after a colpotomy has been made. Obviously, the uterus cannot be manipulated by instruments that would tend to penetrate or otherwise damage it. Likewise, in many cases, the patient's bowel, small intestines, large intestines, bladder, stomach, liver, kidneys and the like may interfere with the surgery. Currently, retractors are used by the surgeon to move the non-target organs away from the surgical site.

In some laparoscopic operations, the patient is placed in what is known as the Trendelenburg position, sometimes at steep angles, in order to keep the non-target organs, such as the bowel and intestines, out of the surgeon's way. By placing the patient in the Trendelenburg position, which involves inclination of the patient's body with his or her head down and legs elevated, the small bowel is caused to glide away from the pelvis, creating a working space within the abdominal cavity. To optimize surgical exposure, an angle of inclination of up to 40 degree may be required. However, prolonged Trendelenburg position significantly increases intracranial pressure and intraocular pressure. In addition to the circulatory effects, steep head-down impacts on the cardiac system due to increased central venous pressure, and on the respiratory system by decreasing total lung volume, pulmonary compliance, and functional residual capacity. Thus, this position with the head lowered causing increased blood flow to the brain is generally undesirable.

Rather than invert the patient to conduct a surgical laparoscopic procedure, many surgeons use some sort of grasper or retractor to support and maintain the non-target organ out of the area of the target surgical organ. Many graspers have distal ends which can puncture or perforate tissue and organs, making their use only attempted by highly trained surgeons and may also require multiple surgeons during the same procedure as one surgeon will be necessary to grasp and maintain the non-target organ out of the area where a second surgeon is performing a procedure on the target organ or tissue. This may result in increased costs to the medical office, hospital, insurance company and ultimately the patient.

Other devices which may be used include clamps such as without limitation bowel clamps. The existing bowel clamps are designed to clamp directly on the bowel during for instance a resection procedure and to cut off that portion of the bowel where the clamp is affixed. The direct clamping or pinching engagement with the bowel wall results in a significant number of drawbacks when being used to retract and support a bowel not going through a resection being intentionally cut or punctured. The engagement of the clamp on the bowel can puncture the bowel wall, which is not catastrophic during a resection where a bowel section is being removed from the body but would be devastating when attempting to move, retract and then return the bowel to a position within the body cavity. Additionally, the movement of the bowel by using the engaged clamp can tear the bowel. This is due, in part, to the minimal surface engagement between the clamp and the bowel wall.

When the bowel wall is compromised, whether by the bowel engaging clamp, graspers or otherwise, numerous complications can arise. First, the contamination of the interior body cavity with the dirty contents of the bowel interior can increase the risk of infection and other adverse effects. Furthermore, once a puncture or tear has occurred, the surgeon has to take appropriate remedial measures. These remedial measures may include further resection of the bowel to include the bowel segment which has been damaged, or separately stitching or otherwise closing the puncture or tear in the bowel to repair the damage caused by the clamp. Either of these remedial measures increases the time of the surgery. Additionally, both of these remedial measures increase the risk of infection and the like to the patient by exposing the contents in the interior of the bowel to the interior of the body cavity. Therefore, this damage to the bowel needs to be avoided. The same is true of the small intestine, large intestine, bladder, stomach, liver, kidneys and the like in that exposing the contents of such organs through a puncture or tear may be devastating to the patient.

Other organs which may need to be moved or retracted during a laparoscopic surgery may also be damaged during the procedure, such as the uterus, liver, stomach, intestines, heart, lungs, glands, etc. Each organ or tissue may be scraped, punctured, bruised or otherwise damages using the conventional retractors, clamps or graspers during laparoscopic surgical procedures to move the non-target tissue or organ while the surgeon works on the target tissue or organ. While some retractors have been devised to include inflatable balloons and the like so to avoid unintentional puncture of the bowel or other organs during surgery, such devices are large and take up space within the small surgical site resulting in obstruction of the view of the surgeon while working on the target organ or tissue.

Thus, there is a need for a device which can be used during laparoscopic surgery to support a non-target body organ (such as without limitation the uterus, bowel or intestines) without trauma to the non-target organ while not decreasing the area of the surgical site and view of the surgeon. A further needs exists for a device which can be used during laparoscopic surgery to support a non-target body organ (such as without limitation the uterus, bowel or intestines) without trauma to the non-target organ while the patient rests in a normal operating position.

Another need exists for a device which can be used during laparoscopic surgery to retract, support and maintain a non-target body organ without trauma to the non-target body organ and without trauma to the target organ or tissue.

Yet a further need exists for a device which can be used to retract, support and maintain a non-target body organ without trauma to the non-target body organ and without trauma to the target organ or tissue while maintaining the level of pneumoperitoneum during the surgical procedure.

It is an object of the present invention to overcome the drawbacks of the prior art. It is a further object of the present invention to provide support for an organ or tissue which can be easily used in laparoscopic or open procedures for engaging and moving the organ or tissue while significantly reducing the risk of damaging the bowel.

A further object of the present invention is to provide a bowel support device such as a sling.

It is yet a further object of the present invention to provide an organ retractor or organ sling which can be easily used in laparoscopic or open procedures for engaging and moving non-target organ, such as a uterus or intestine, while significantly reducing the risk of damaging to the non-target organ.

Accordingly, there remains a need in the art for the inventive surgical support device, the device's method of use, and a kit containing the inventive device. The present invention provides a solution for these needs and other needs.

The present invention has been made to solve the above problems occurring in the prior art and other needs in regard to surgical instruments and methods of treatment.

### SUMMARY OF THE INVENTION

The above objects are achieved by a surgical support device according to the present invention. In accordance with the invention a device capable of retracting, supporting and maintaining a body organ, such as the uterus or bowel, includes a band or sling made of a biologically inert material which either supports or retracts the organ away from the surgical site or fans out when a number of supports are employed at the mouth of the band or sling portion. The band or sling is large enough to support the non-target body organ to be retracted when the band or sling is opened after it has been deployed out of the housing or cannula. After the band or sling is passed through the cannula, the mouth of the band or sling is positioned over the non-target body organ and the device is manipulated and moved such that the non-target body organ is moved and retracted to the desired position. The device is then stabilized such that the retracted non-target body organ is secured. In certain instances one end of the band or sling may be grasped with a laparoscopic grasper to further stabilize the band or sling and contained retracted non-target body organ. In other circumstances the surgeon can place a temporary suture into one end of the band or sling to secure the sling containing the non-target body organ in a more secure retracted position within the body cavity.

When the surgical procedure or operation is complete on a target organ on tissue, the band or sling is manipulated to release the contained non-target body organ or tissue. If a grasper has been employed to further secure one end of the band or sling, such grasper is released. If a suture has been employed to further secure one end of the band or sling, the suture is cut and the band or sling removed from the non-target body organ or tissue which returns to its normal position. The band or sling is next withdrawn through the cannula by force and the entire device removed from the applicable body cavity.

An inventive knob mechanism may be included within the inventive device as one deployment means to deploy the band or sling.

### DESCRIPTION OF THE DRAWINGS

The above and other advantages of the present invention will become readily apparent with reference to the following detailed description when considered in conjunction with the accompanying drawings which are incorporated in and constitute part of this specification, are included to illustrate and provide a further understanding of the devices and related methods of the invention. Together with the description, the drawings serve to explain the principles of the invention, wherein:

FIG. 1 is a perspective view of one embodiment of the present inventive device in a non-deployed state.

FIG. 2 is perspective view of another embodiment of the present inventive device in a non-deployed state including a disposable protective sheath.

FIG. 3 is a perspective view of another embodiment of the present inventive device in a non-deployed state including a slightly curved housing and a protective sheath.

FIG. 4 is an exploded view of the distal end of the housing of the present inventive device in a non-deployed state.

FIG. 5 is an exploded view of FIG. 4 of the distal end of the present inventive device in a non-deployed state.

FIG. 6 is an exploded view including a cut-away internal view of the distal end of the present inventive device in a non-deployed state.

FIG. 7 is an exploded view including a cut-away internal view of the distal end of the present inventive device in a non-deployed state.

FIG. 8 is a perspective view of one embodiment of the present inventive device with a sling in a deployed state.

FIG. 9 is perspective view of another embodiment of the present inventive device with a band in a deployed state.

FIG. 10 is a perspective view of another embodiment of the present inventive device including a slightly curved housing and a band or sling in a deployed state.

FIG. 11 is a side exploded view of the distal end of another embodiment of the present inventive device in the deployed state.

FIG. 12 is top view of an exploded view of a cut-away internal view of the distal end of one embodiment of the present inventive device of a sling in the deployed state.

FIG. 13 is an exploded perspective view of the knob mechanism of one embodiment of the present inventive device in a deployed state.

FIG. 14 is an exploded view of a cut-away internal view of the knob mechanism of one embodiment of the present inventive device.

FIG. 15 is an exploded internal view of one embodiment of the present invention including an inventive knob activator and deployment mechanism.

FIG. 16 is a side perspective exploded view of one embodiment of the present invention including a supporting means connector which pivots around a pivot pin from the unactuated position to a deployed position.

FIG. 17 is an exploded internal view of one embodiment of the present invention including a blunt distal end tip and supporting means connector which pivots around a pivot pin from the unactuated position to a deployed position.

FIG. 18 is an exploded side view of one embodiment of the present invention including a heat shrink tube on the housing adjacent to the proximal end of the aperture.

FIG. 19 is an exploded internal view of the embodiment of FIG. 20 showing a tab and seal on a proximal end of the aperture within the hollow housing where the band deploys.

FIG. 20 is an exploded view of one embodiment of the present invention including two supporting means layered and connected together via a rivet and an inner tube within the hollow housing.

FIG. 21 is a perspective view of another embodiment of the present inventive device including a push and pull rod activator with the device in a non-deployed state.

FIG. 22 is a perspective view of another embodiment of the present inventive device including a pistol grip deployment mechanism with the supporting means and sling in a non-deployed state.

FIG. 23 is a top view of a kit including an obturator and one embodiment of the present inventive surgical support device in a non-deployed state.

### DETAILED DESCRIPTION OF THE INVENTION

Reference will now be made in detail to the present invention, examples of which are illustrated in the accompanying drawings.

In accordance with one aspect of the invention, as indicated above, the inventive surgical instrument device is provided which has utility with various different body organs or tissue which may need to be retracted and supported during open or laparoscopic surgery. Typically, such body organs during abdominal procedures include the bowel, uterus, ovaries, fallopian housings, small intestine, large intestine, liver, kidney, and any other body organs. The inventive surgical device may also be employed in other surgical procedures so as to retract other body organs within a body cavity out of the area (and view for the surgeon) of the surgical site and to support such non-target body organs or tissue during the procedure.

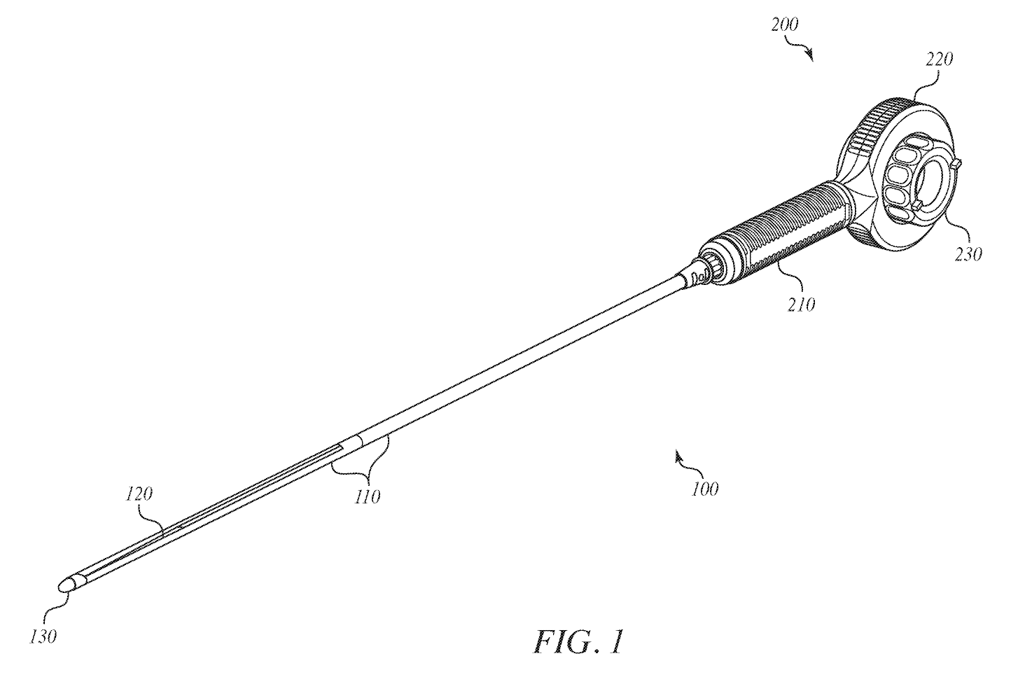

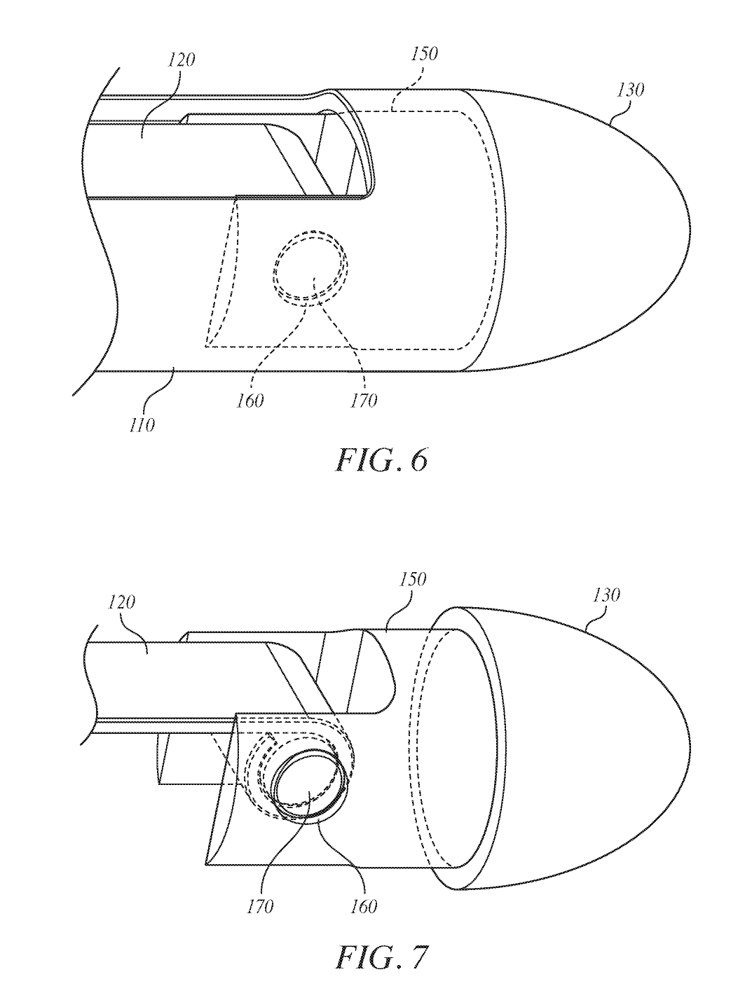

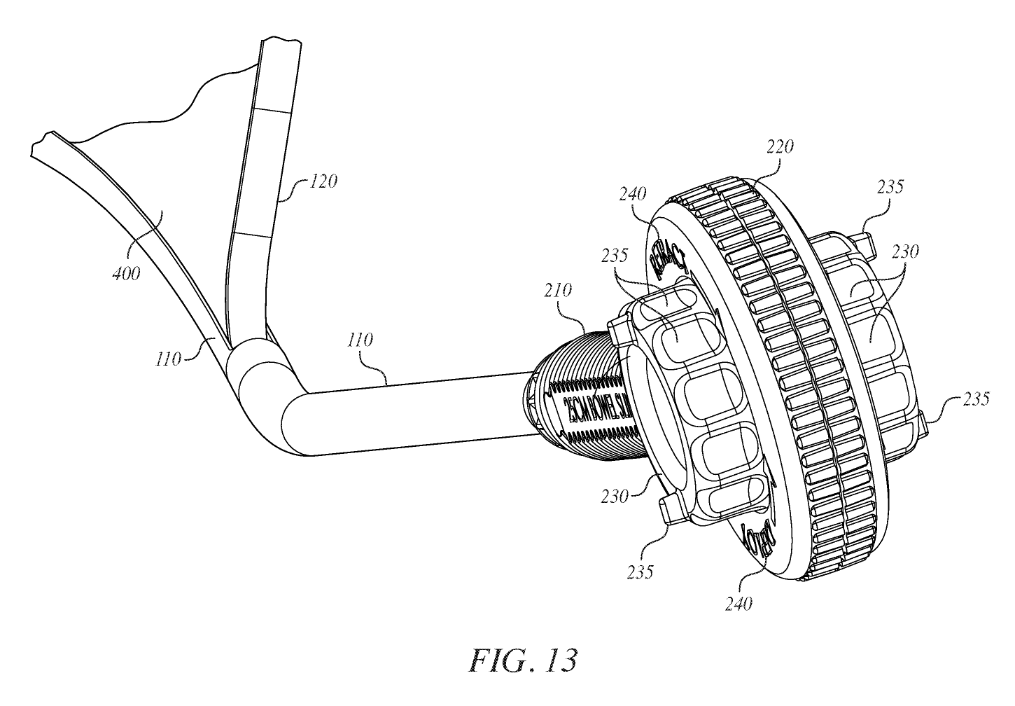

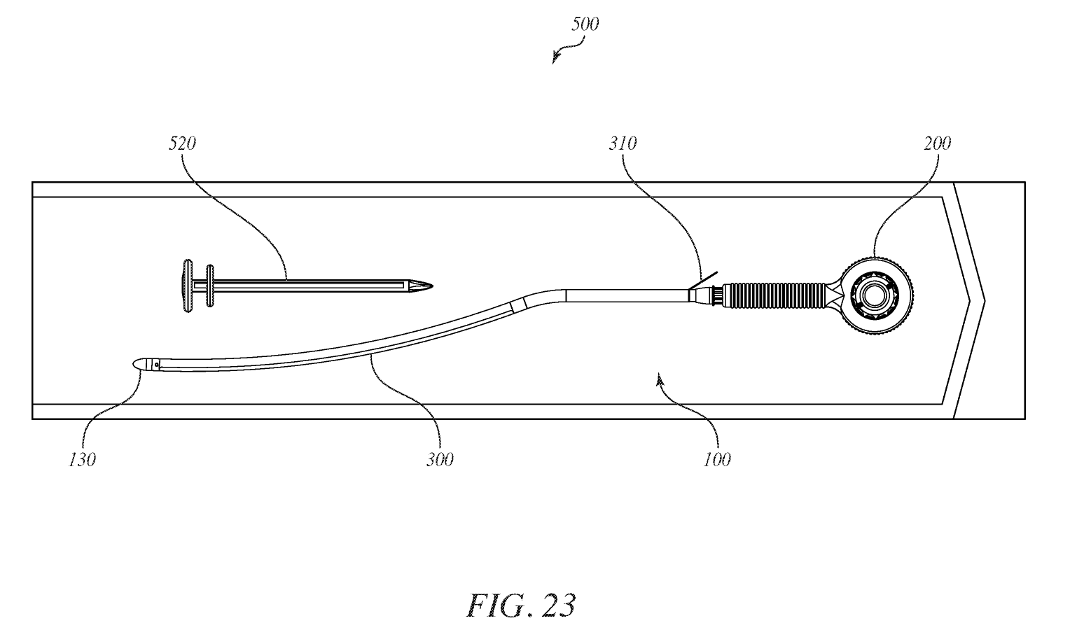

As shown in FIGS. 1 through 23, a surgical instrument device according to the invention comprises an elongated housing, an activator or deployment means, and band or sling which is attached to a flexible supporting means. The device includes a hollow housing through which the supporting means and band or sling are housed in an unactivated state and then deployed into an activated state out of an aperture in the housing so as to retract or support the organ or tissue during the surgical procedure. Once the surgical procedure is completed or partially completed such that the retracted organ or tissue may be returned to its original position within the body cavity, the supporting means and band or sling are deactivated and retracted or withdrawn back into the housing. This activation and deactivation may be repeated numerous times throughout the surgical procedure as the inventive surgical device may be used to retract, support and maintain one organ or area of tissue and then moved to any other organ or area of tissue and so on throughout the surgical procedure.

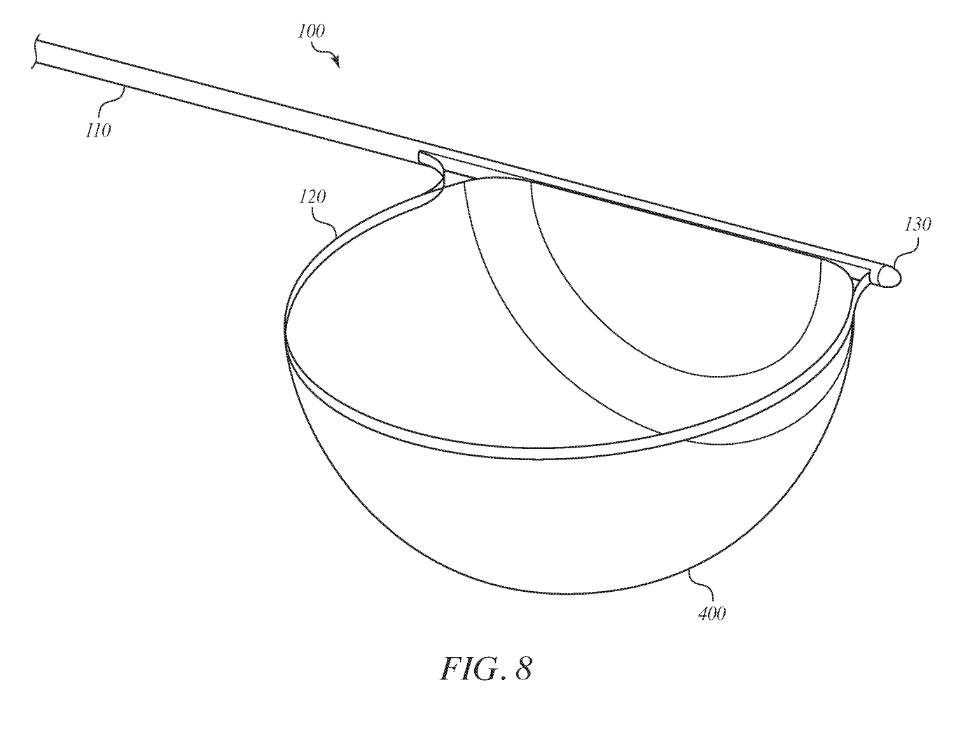

For the purposes of explanation and illustration, and not limitation, in accordance with the invention, exemplary embodiments of a surgical device are illustrated in FIGS. 1 through 23, with the inventive surgical device shown show in the non-deployed position in FIGS. 1 through 8 and 21 through 23 and in the open and actuated deployed position in FIGS. 8 through 20. In accordance with these examples, the inventive surgical device 100 includes a housing 110 with an aperture 140 from which a supporting means 120 is deployed by the activator or deploying means 200. The housing 110 contains the inactivated, unopened and non-deployed sling 400 or band 500 which is connected to and supported in the activated and opened state by the flexible supporting means 120. The activator or deployment mechanism 200 may be one of many known deployment means such as a push rod or various grips, and in one embodiment of the present invention includes an inventive knob rotator mechanism including a distal end grip 210 connected to the proximal end of the housing 110 and a knob grip 220 and actuator 230. The means which has direct contact with the organ or tissue so as to retract and support the organ is referred to herein as a sling 400 or a band 500 with the difference between the two, for exemplary purposes only, is whether the material includes sag or a pouch like portion to maintain and support the organ in which case the material with a pouch is referred to as a sling 400 and the material without a pouch is referred to as a band 500. Each of the terms sling 400 and band 500 are interchangeable within the disclosure of the invention and are used merely to illustrate the amount of material which is used to support and maintain the retracted organ or tissue.

As indicated above, the material and dimensions of the band 500 or sling 400 must be such that it can be inserted through a housing 110 commonly used in laparoscopic surgery. Typically, such housings 110 or cannulas have inner diameters of from about 2.5 millimeter to about 50 millimeters, though smaller diameters of under 2.5 millimeters may be employed. The band 500 or sling 400 material described above can be folded within the cannula 110 and when deployed unfolds. The inventive surgical device may be self-inserting or it may be inserted within a trocar, which trocar diameter may affect the diameter of the housing 110.

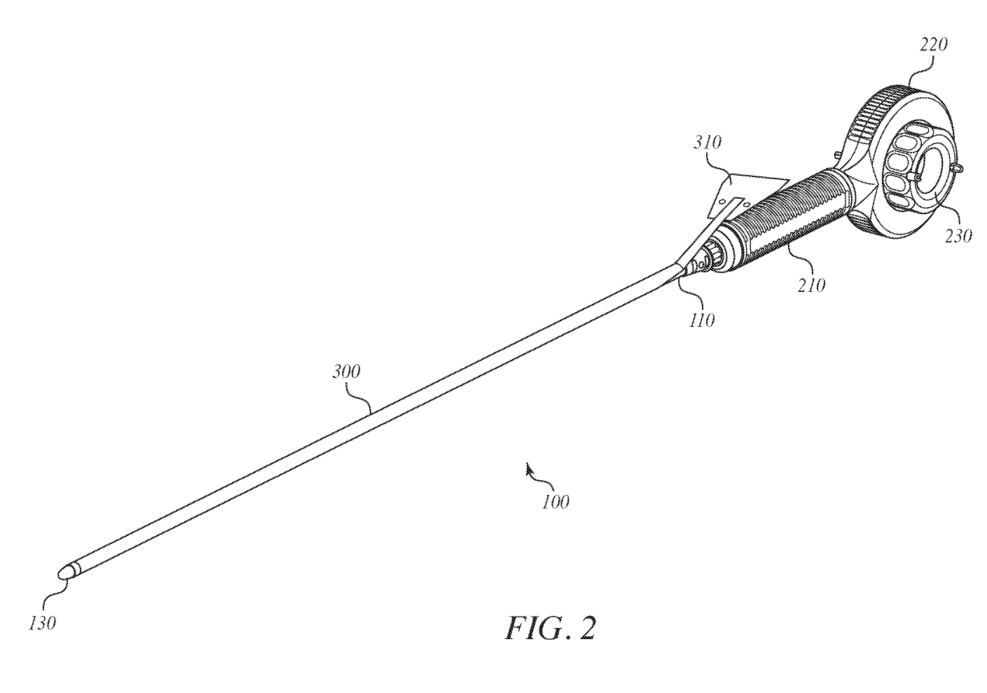

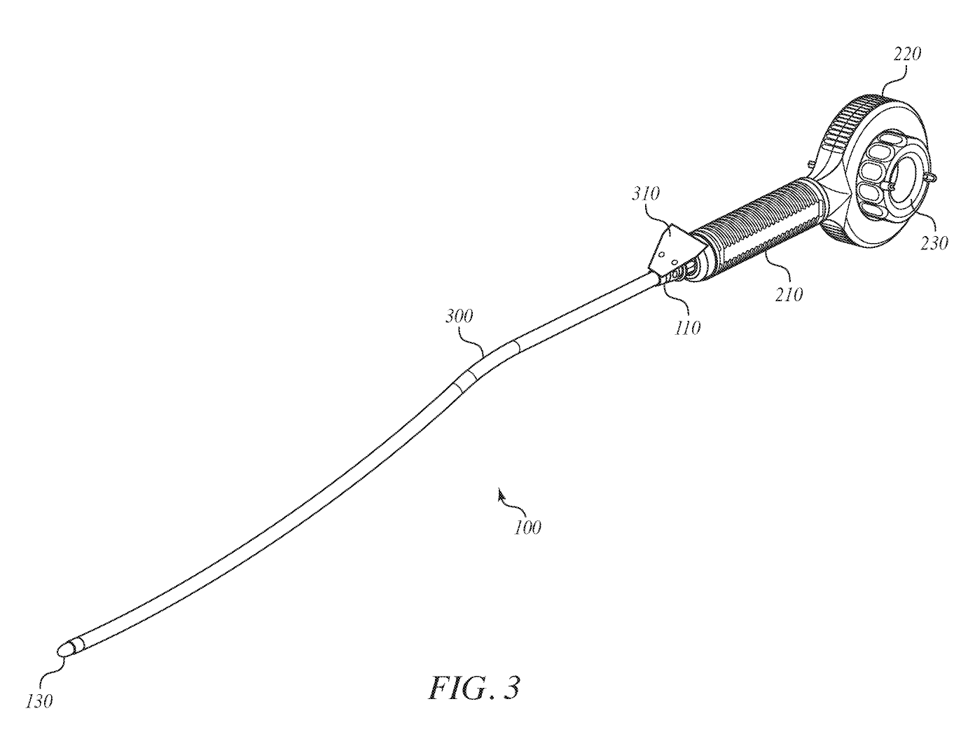

As shown in FIG. 1, the inventive surgical device 100 is in an unactuated state such that the supporting means 120 is retracted within the housing 110 and the sling 400 or band 500 is not shown as it is stored or folded behind the supporting means 120. Based on the diameter of the housing 110, the housing 110 itself may be in a straight linear shape as shown in FIGS. 1 and 2 or slightly bent as shown in FIGS. 3, 13 and 21. The shape of the housing 110 may be affected by the length of the housing 110 based on the location of the surgical site and location of the organ or tissue to be retracted, supported and maintained by the inventive surgical device 100. The length of the housing 110 may also be affected by the size of the sling 400 or band 500 in that the aperture 140 would be of a certain length and size relative to the length and size of the sling 400 or band 500. The housing 100 length may also be affected by the size of the patient such as an infant, child, adult, or obese patient. Other factors may affect the length, shape, curve or straightness, and diameter of the housing 110.

In accordance with these examples, the inventive surgical device 100 includes a housing 110 which may be a cannula or housing having a diameter, for example 3 mm or 10 mm, in a range of about 1 mm to about mm, with a length in a range of about 10 mm to about 1000 mm. The housing 110 may be straight or slightly curved at a point 115 with a curved portion 115 located at a position on the housing 110 approximately where the housing 110 would be located outside of the body cavity, or at another location as determined by the size of the patient and distance within the body cavity and other factors. The housing 110 (or cannula) of the inventive surgical device 100 may be comprised of any material compatible with the human body as a portion of it will be within the body cavity when in use. The housing 110 is preferably comprised of a metal such as stainless steel and is preferably coated with a shrink wrap plastic such as shrinkable polyester, polyolefin, polyethylene, or polyvinyl chloride of a grade suitable for use in surgical procedures.

As shown in FIGS. 1 and 3, a certain portion of the housing 110 will remain outside of the body cavity, whereas a certain portion of the housing 110 within the distal end of the housing 110 will be placed within the body cavity as that portion of the housing 110 contains the supporting means 120 and the sling 400 or band 500 in the inactivated state within the distal end of the housing 110. In comparison as shown in FIGS. 3, 9, 11-12 and 17 the housing 110 is bent at a certain point so that the distal end containing the supporting means 120 and the sling 400 or band 500 in the inactivated state may be placed within the body cavity in one linear position whereas the proximal end of the housing 110 remains outside of the body cavity within the trocar, obturator or access port. In this second embodiment of the inventive surgical device the length of the aperture 140 is longer as is the supporting means 120 and the sling 400 or band 500 so as to retract and support a larger organ or tissue area.

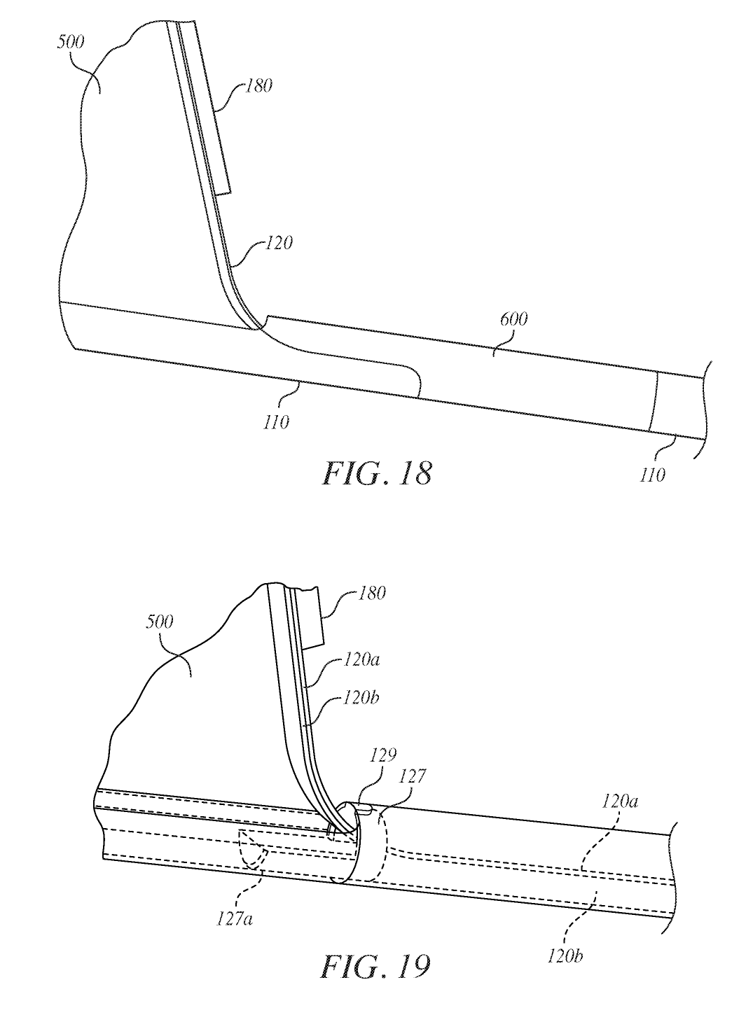

A distal end of the housing 110 has a tip 130 which tip 130 is configured in one embodiment to be self-inserting and have a non-blunt shape with another embodiment having a blunt shape 135. The distal end tip 130 in FIGS. 1 through 8 is shown as one embodiment in the shape of bullet nose, though any tip may be employed. In other embodiments of the present invention the distal end tip 130 may be curved, hooked, pointed, bull-nosed, combinations thereof, or any other non-blunt shape. As shown in FIGS. 18-22 the distal end tip 135 has a blunt shape so to avoid any inadvertent nick or puncture of any surrounding organs or tissue.

The distal end tip 130, 135 is connected to the housing 110 by a connecting means such as a faster, hinge, welding, soldering, crimping, adhesives or another suitable technique, as desired. In an alternate embodiment of the present invention (not shown) the distal end tip 130 may be formed as part of the flexible supporting means, on the distal end thereof, and need not be attached to the housing. The distal end tip 130, 135 may be comprised of any material compatible with the human body as it will be within the body cavity when in use. The distal end tip 130, 135 is preferably comprised of a plastic such polyester, polyolefin, polyethylene, or polyvinyl chloride, or may be a metal such as stainless steel and may be coated with a shrink wrap plastic such as shrinkable polyester, polyolefin, polyethylene, or polyvinyl chloride of a grade suitable for use in surgical procedures.

The sling 400 or band 500 is connected on one side 405 or 505 to the inner diameter of the housing 110 corresponding to the length of the aperture 150. The connection of the sling 400 or band 500 to the inner diameter of the housing 110 may be made with an adhesive or other bonding material such as glue, adhesives, curing or other means suitable to prevent the sling 400 or band 500 from separating from the housing 110 upon deployment and the force necessary to retract, support and maintain the relevant organ or tissue during use.

The sling 400 may be comprised of any material which is biocompatible with the body as it will be deployed within the body cavity and in direct contact with an organ or tissue as such organ or tissue is retracted and supported by the deployed sling 400. In one embodiment of the present invention the sling 400 is comprised of a polymer mesh material with small openings that do not damage the retracted and supported tissue or organ. For instance, the sling 400 may be comprised of elastomeric or polymeric material such as but not limited to polyurethane, polyethylene, polypropylene, vinyl, latex, polymers, plastics, or combinations thereof. The material of the sling 400 should have sufficient strength so as not to tear when in use given the force needed to retract, support and maintain the relevant organ or tissue during use in a surgical procedure. In general the sling 400 may be used for larger organs or tissue to be retracted and supported during a surgical procedure as it may have a pouch or other shape with can maintain an organ or tissue during use. As shown in FIG. 8 the sling 400 has a pouch into which the organ or tissue may rest and be contained during retraction and support in use.

The band 500 may be comprised of any material which is biocompatible with the body as it will be deployed within the body cavity and in direct contact with an organ or tissue as such organ or tissue is retracted and supported by the deployed band 500. In one embodiment of the present invention the band 500 is comprised of a polymer material which is taut when in the activated deployed state. For instance, the band 500 may be comprised of elastomeric or polymeric material such as but not limited to polyurethane, polyethylene, polypropylene, vinyl, latex, polymers, plastics, or combinations thereof. The material of the band 500 should have sufficient strength so as not to tear when in use given the force needed to retract, support and maintain the relevant organ or tissue during use in a surgical procedure. In general the band 500 may be used for smaller organs or tissue to be retracted and supported during a surgical procedure, or in pediatric patients or in smaller body cavities in adult patients.

The sling 400 or band 500 may be comprised of film, elastomeric or polymeric material such as but not limited to polyurethane, polyethylene, polypropylene, vinyl, latex, polymers, plastics, or combinations thereof. Any material which is compatible with the body may be used for the sling 400 or band 500. Further, the sling 400 or band 500 is comprised of a material which is preferably transparent or semi-transparent so that the operator can observe the non-target specimen of tissue or an organ received in the sling through an endoscope with naked eyes. The sling 400 or band 500 may be of a dimension suitable for encompassing the non-target organ such as the bowel or uterus. The shape of the sling 400 or band 500 may be any geometric shape and may be dependent on the shape of the organ or tissue to be retracted, supported and maintained while in use. The size of the sling 400 or band 500 may be It is contemplated that the sling 400 or band 500 may be made in small, medium and large sizes depending, of course, on the size of the non-target body organ. The size and area of the sling 400 or band 500 may be of a dimension ranging in a length of about 2 mm to about 50 mm and a width of about 2 mm to about 50 mm, including from a length of about 5 mm to about 30 mm and a width of about 3 mm to about 10 mm. Other shapes may be employed for the sling 400 or band 500 such as circular, oval, a “D” and the like with a diameter in the range of about 2 mm to about 50 mm.

The sling 400 or band 500 may be comprised of any biocompatible material, such as mesh. The material may be fabricated from any of a number of biocompatible materials such as nylon, polyethylene, polyester, polypropylene, fluoropolymers, copolymers thereof, combinations thereof, or other suitable synthetic material(s). The material may be, for example, a synthetic material that is absorbable by the patient's body. Suitable absorbable synthetic materials include polyglycolic acid, polylactic acid, and other suitable absorbable synthetic materials. The mesh material may be fabricated from one or more yarns, which yarns may be made from one or more materials. The mesh may be produced according to numerous fabrication processes, and may be designed to permit rapid tissue revascularization and tissue in-growth by having large interstitial spaces. For example, each yarn of the mesh may have void areas between yarn filaments and the fabrication process may create crevices.

The sling 400 or band 500 is connected on one side 405 or 505 to the inner diameter of the housing 110 and on all other sides to a flexible supporting means 120. The flexible supporting means 120 may be comprised of biocompatible material as it will have contact with the organ or tissue within the body cavity upon deployment and activation. The supporting means 120 may be comprised of any flexible materials which is capable of compression when housed within the housing 110 and capable of forming an open position for the sling 400 or band 500 once deployed and actuated outside of the aperture 140 of the housing 110. The supporting means 120 may be a spring resilient material to give it a tendency to form a circle, oval, “D” shape or other shape in a fully deployed position. When the surgical device 100 is no longer needed and it is ready to be withdrawn from the body cavity, the supporting means 120 is deactivated and retracted back into the housing 110 and collapses under manual force against spring bias to fit within the housing 110. For instance, the supporting means 120 may be comprised of a flexible metal such as stainless steel, may be a high strength stainless steel alloy, titanium, nitinol, steel spring alloys, metal alloys, plastics, combinations thereof. In general, the supporting means 120 may be comprised of any material that can be stored in a deformed shape, is resilient, and can be returned via compression or other force to an initial or near initial shape.

In one embodiment of the present inventive surgical device 100, the sling 400 is comprised of a laser cut polyester surgical mesh with certain a sag, in one embodiment about 4 cm sag is depicted but any reasonable sag is acceptable. The supporting means 120 onto which the sling 400 is connected may be a resilient materials, such as a wire or stainless steel band. In one embodiment of the present invention the supporting means 120 is inserted through a series of laser cuts slots in the mesh sling 400 so as to connect the mesh sling 400 to the supporting means 120. The supporting means 120 in this embodiment slides through the slots in the sling 400 and is connected. Further, at the point of these connections between the slots in the sling 400 and the supporting means 120 a further connection is made via a heat shrink 180. Instead of a heat shrink 180 a RF weld may be applied or other connecting means may be employed. The heat shrink or RF weld provides further connection of the supporting means 120 and sling 400 as well as preventing atraumatic edges of the sling 400 because the sling 400 may have slight protrusion where the slots are connected to the supporting means 120. Further, the sling 400 is connected to the inner portion of the housing 110 along the back portion of the aperture via a connecting means such as an adhesive, sealing or welding. The connecting means may be a RF weld or UV bonding.

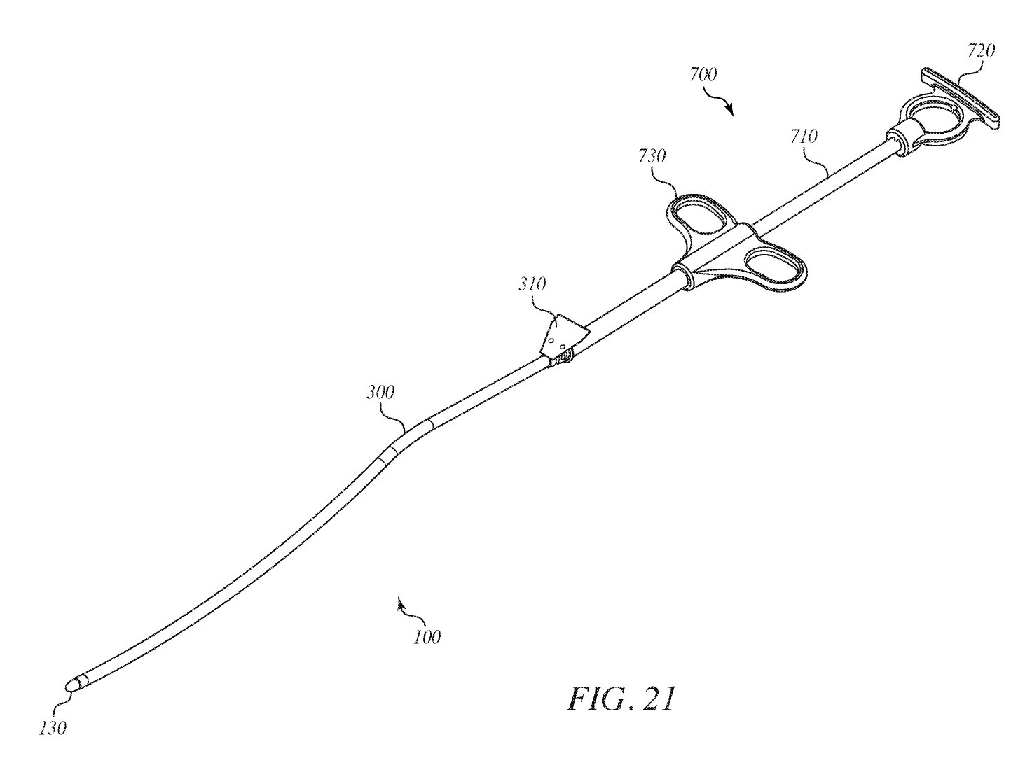

A deploying mechanism 200 is also included with certain portions of the surgical device 100. The activator or deployment mechanism 200 is located opposite to the distal end tip 130 of the hollow housing 110. The activator or deployment mechanism may be, but is not limited to, a push rod, loop grip mechanism, pistol grip handle mechanism, twist mechanism, thumb grip mechanism, flexible ring member mechanism, a push/pull mechanism, or combinations thereof. A proximal end of the supporting means 120 is connected to a distal end of the activator or deployment mechanism 200 such that when the activator or deployment mechanism 200 is engaged and activated it creates a force against or on the proximal end of the supporting means 120 and forces the distal end of the supporting means 120 out of the aperture 140 of the housing 110 and thus activates and forces out the sling 400 or band 500 which is connected to the supporting means 120. At a later time during the surgical procedure the surgeon may deactivate the inventive surgical device 100 via the activator or deployment mechanism 200 and thus the supporting means 120 and the attached sling 400 or band 500 are withdrawn and retracted back into the aperture 140 and the housing 110. For instance, if the proximal end of the supporting means 120 is connected to an activator or deployment mechanism 200 which is a push and pull rod (as shown in FIG. 21), when the rod of the activator or deployment mechanism 200 is pushed in a downward direction it would force the supporting means 120 from within the housing 110 out of the aperture 140 and thus unfold and force out the connected sling 400 or band 500 resulting in a deployed and activated surgical; device 100 and the opposite would occur if the rod of the activator or deployment mechanism 200 is pulled in an upward direction it would pull the supporting means 120 from outside the housing 110 back into the aperture 140 and thus retract back and withdraw the connected sling 400 or band 500 into the aperture 140 and back into the housing 110 resulting in a non-deployed and unactivated surgical device 100.

In one embodiment of the present invention as shown in FIGS. 1 through 3, 9 through 10, 13 through 15 and 23, the deploying mechanism 200 includes a knob mechanism 210, 220 and 230. In this embodiment a proximal end of the supporting means 120 is connected to the knob mechanism 200 such that when the knob mechanism 200 is moved in one direction it forces the supporting means 120 from within the housing 110 out of the aperture 140 and thus unfolds and forces out the connected sling 400 or band 500 resulting in a deployed and activated surgical device 100 and the opposite would occur if the knob mechanism 200 is moved in an opposite direction to then pull the supporting means 120 from outside the housing 110 back into the aperture 140 and thus retract back and withdraw the connected sling 400 or band 500 into the aperture 140 and back into the housing 110 resulting in a non-deployed and unactivated surgical device 100.

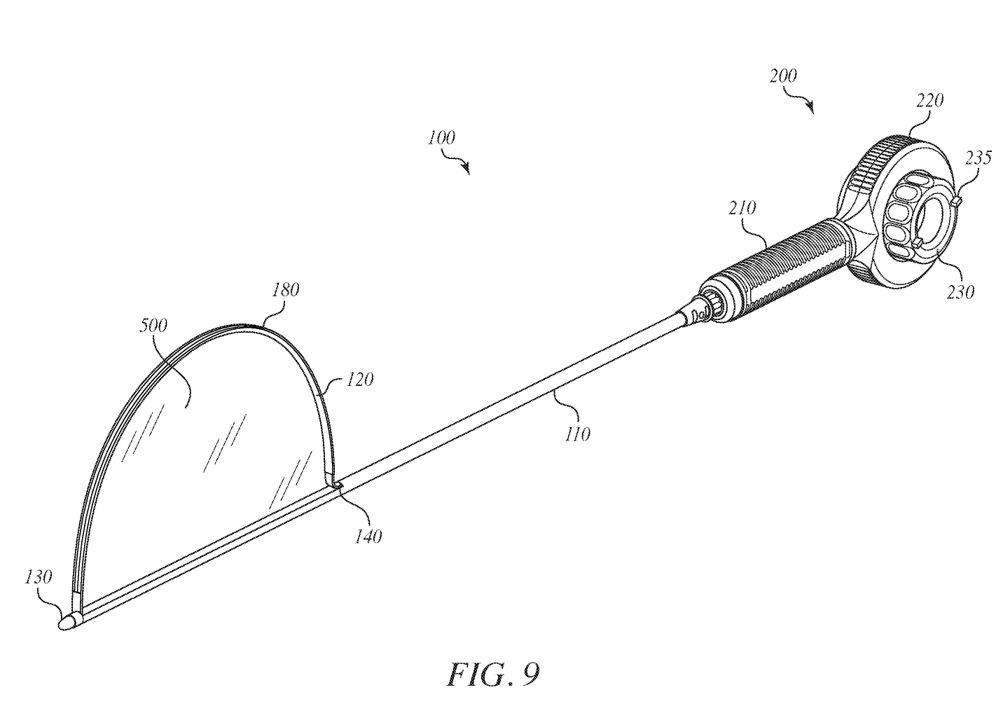

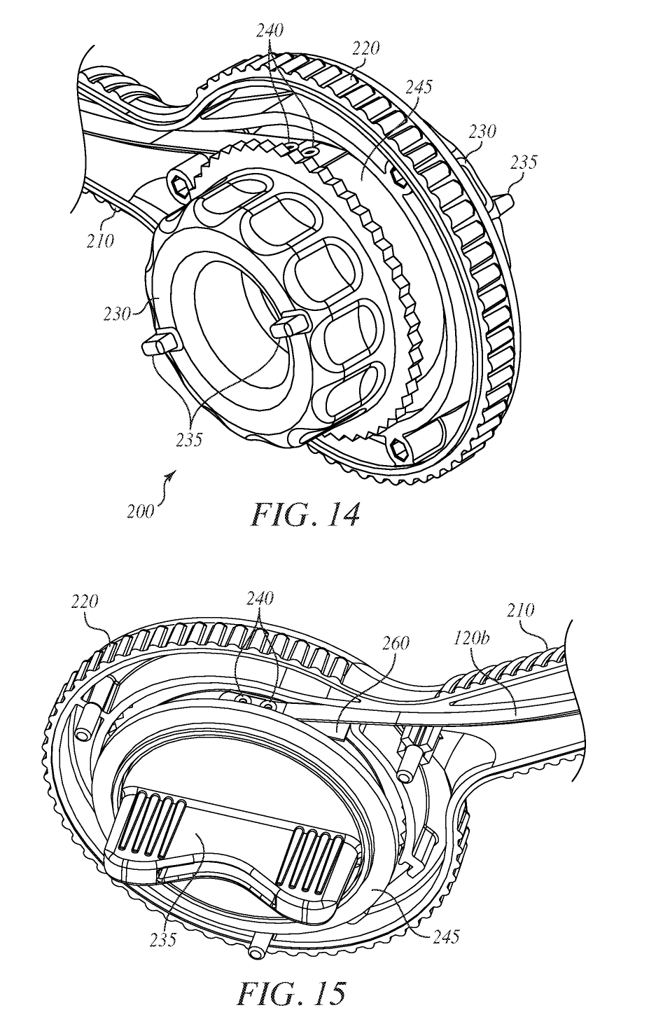

The knob rotator mechanism is shown in more details in FIGS. 9 and 13 through 15 with the knob rotator mechanism 200 including in one embodiment of the present invention, a finger grip 210 connected to the proximal end of the housing 110. A knob 220 also includes groves for gripping or grabbing by the surgeon when in use. Two side rotating finger knobs 230 are located on each side of the knob rotator mechanism 200 so that the surgeon can use either side during the surgical procedure. The side rotating finger knobs 230 include indents for better gripping. The side rotating finger knobs 230 further includes in this embodiment of the present invention two extending features 235 for better grasping and rotation of the knob mechanism 200 by the surgeon in use. As shown in FIGS. 14 and 15, the supporting means 120 extends through the housing 100 up to the knob rotator mechanism 200 so that no rod is needed as the supporting means 120 is activated and retracted by spooling around the inside of the knob 220 as activated and retracted with the turning in one direction versus an opposite direction of the knobs 230. The supporting means 120 is connected to the spool 245 via a connecting means such as rivets 240. The surgeon has both a visual and audio awareness of the activation and retraction of the supporting means 120 via printed indicators 240 of the direction for deploying and retracting the supporting means 120 on the knob 220 as well as the clicking sound created by an inner spring 250 within the knob 220, 230. As shown in FIG. 15 of another embodiment of the inventive knob mechanism on the inventive support device, the device includes two layered supporting means 120a, 120b with an inner supporting means 120b connected to the spool 245 via a connecting means such as one or more rivets 240. During use the inner supporting means 120b starts in an unactuated state within the hollowing housing 110 with its proximal end connected to the spool 245 via one or more rivets 240 and becomes activated by unwrapping from the spool 245 and extending out of the aperture 140 into an activated position such that the attached sling 400 or band 500 unfolds from within the hollow housing 110 and can now retract and support an organ or tissue. At a later time the activator is reversed and the supporting means 120 is retracted by turning the knob 220 and the inner supporting means 120b wraps around the spool 245 causing the supporting means 120 and sling 400 or band 500 to retract back into the aperture 140 in the hollow housing 110. A rotational stop 260 may also be included so as to prevent over winding or spooling of the inner supporting means 120b around the spool 245. Other features may also be added.

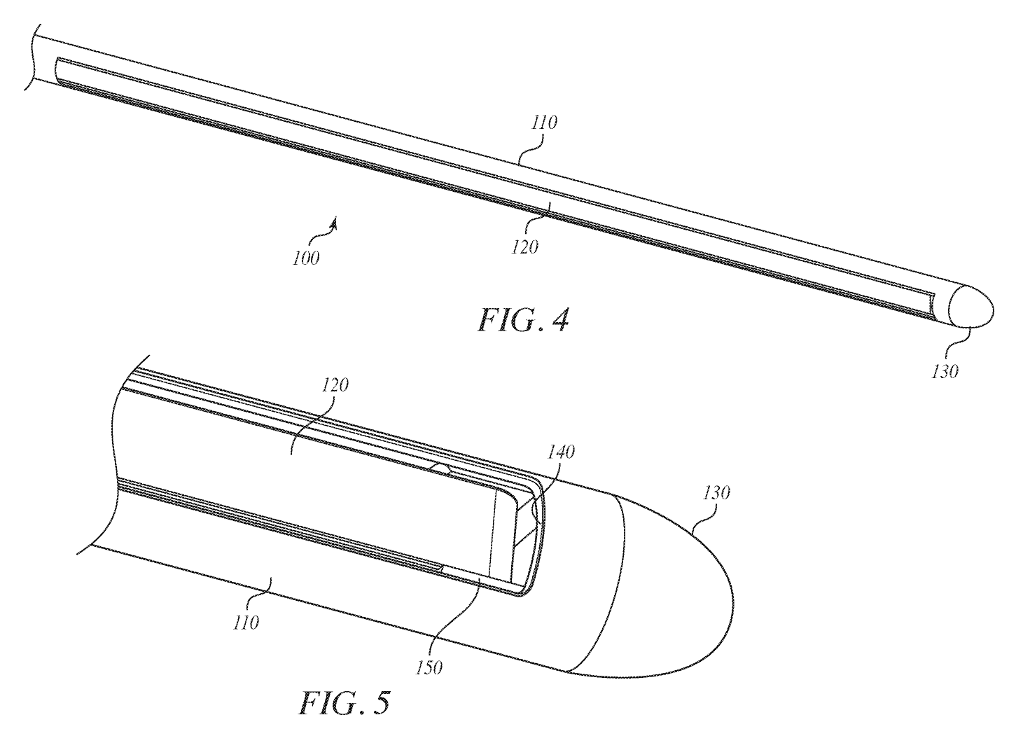

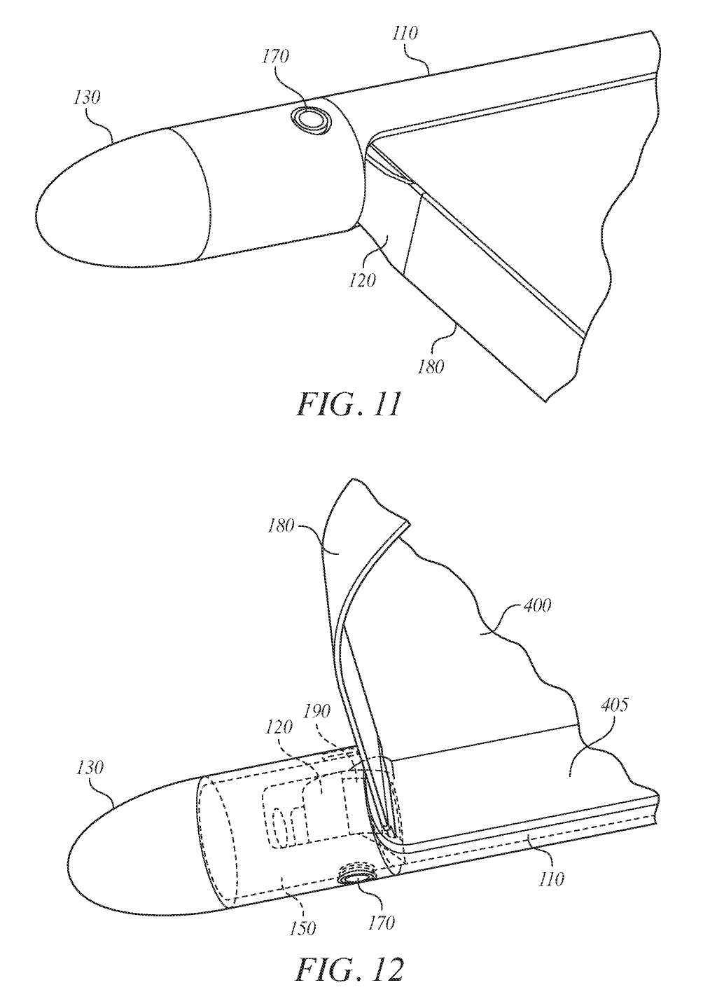

FIGS. 4 through 7, 11 through 12, and 16 through 20 are various close up, exploded and cut-away views of the distal end of the inventive surgical device 100 including the distal end of the housing 110, the distal end tip 130, the aperture 140 through which the supporting means 120 is deployed with the sling 400 or band 500. As shown in FIGS. 11 and 12, the supporting means 120 is connected to a pivot pin 170 via the supporting means 120 forming a loop around the pivot pin 170. The pivot pin 170 is located within an aperture 160 within a bushing 150. The pivot pin 170 is also connected to the housing 100. The bushing 150 is connected to the distal end tip 130. The bushing 150 acts to deploy and retract the supporting means 120.

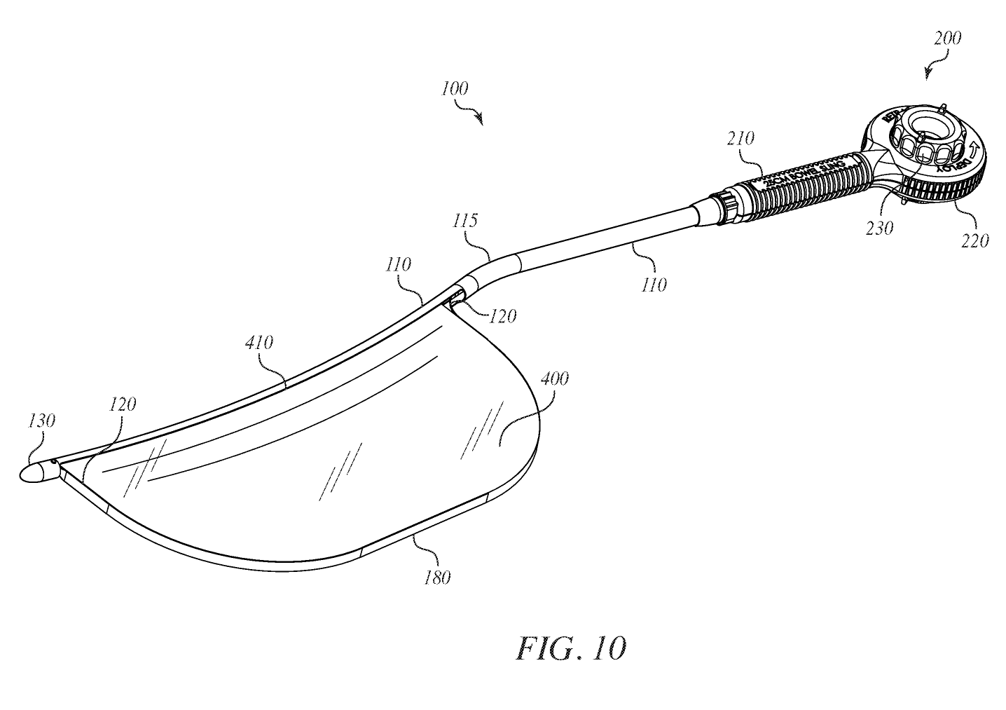

In another embodiment of the present invention a band 500 is used instead of a sling 400. As shown in FIG. 10 the band 500 may be comprised of a polyurethane die cut film which in manufacture of the inventive surgical device 100 is folded back over itself and around the supporting means 120 so that a double layer is taut to form a D shape and form the retracting means. The band 500 is connected to the supporting means 120 such as an adhesive, sealing or welding. The connecting means may be a RF weld or UV bonding. Further, the band 500 is connected to the inner portion of the housing 110 along the back portion of the aperture via a connecting means such as an adhesive, sealing or welding. The connecting means may be a RF weld or UV bonding or other known connecting means.

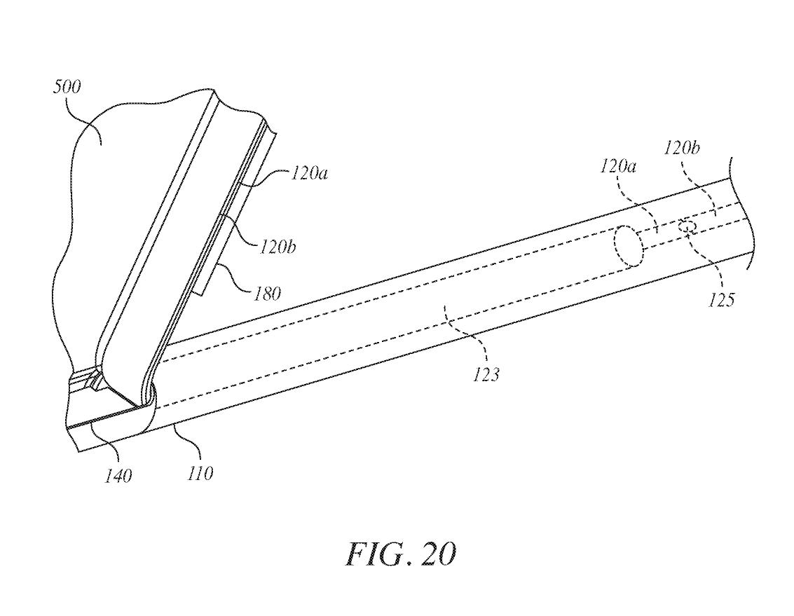

Another embodiment of the present invention is shown in FIGS. 15, 19 and 20 including a multiple of supporting means 120. In this embodiment two supporting means 120a, 120b are shown though additional layers of supporting means 120 may be employed. The two layers of supporting means 120 are an outer supporting means 120a and an inner supporting means 120b. In this embodiment the inner supporting means 120b is connected on a proximal end to the knob mechanism 200 via a connecting means such as a rivet 240 with the spool 245 while the distal end of the inner supporting means 120b is connected to the distal end of the outer supporting means 120a via a connecting mean such as rivet 125 located within a proximal end of the hollowing housing 110. Other connecting means are possible such as adhesives, fasteners, screw, bolts, bonding, welding and the like or combinations thereof. The outer supporting means 120a and the inner supporting means 120b may also be laminated together to provide torsional strength for manipulating and supporting the organ and tissue being retracted, supported and maintained in a position during the surgical procedure, while still maintain flexibility in the opposite direction for deployment of the supporting means 120a, 120b from the inner diameter of the hollowing housing 110. Within the hollow housing 110, as shown in FIG. 20, is also located a tube 123 or other hollow housing to prevent the inner and outer supporting means 120a, 120b from galling against the hollow housing 110 upon deployment and retraction of the supporting means 120a, 120b out of and back into the aperture and hollow housing 110. As shown in FIG. 18 a proximal end of the sling 400 or band 500 may be wrapped partially around the outer diameter of the hollow housing 110 via an adhesive or bonding to provide support and strength. A further feature include a heat shrink tube 600 over such wrapping of the proximal end of the sling 400 or band 500 to prevent peeling of the sling 400 or band 500 from the outer diameter of the hollow housing 110 when the inventive device 100 is removed from the body cavity or a trocar (not shown). Another feature as shown in FIG. 19 is a seal 129 so as to reduce the loss of pneumoperitoneum, provide rotational alignment of the seal 129 and tube 123, and to resist torsional forces. The seal 127 supports the supporting means 120 (or 1202, 120b) laminates to resist twisting when manipulating the organ or tissue during deployment. The seal 127 may include a tab 129 to connect with the housing 110 and may be connected to the inner diameter of the housing at a location shown as 129a with any known adhesive, glue, bonding, welding or the like.

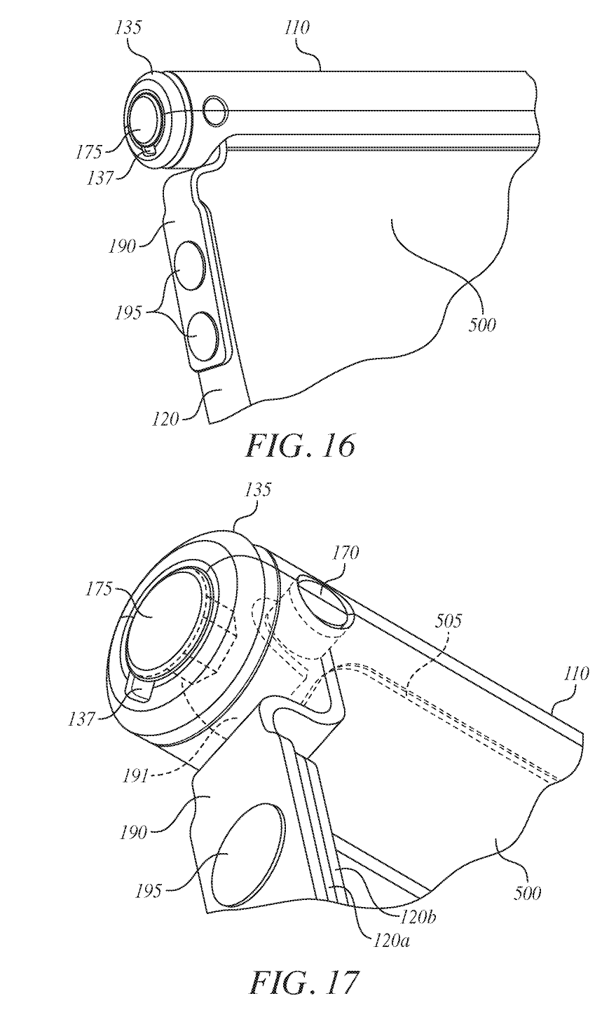

Further features of the inventive knob mechanism 200 within the inventive surgical support device 100 include the blunt tip 135 which houses a portion of the folded and non-deployed sling 400 or band 500 which in one embodiment is wrapped around the inner portion of the distal end tip 135 and connected to a plug 175 via connecting means such as adhesive, glue or other means. As shown in FIGS. 16 and 17 the distal end tip 135 may further include an aperture 137 such as a hex hole into which the adhesive may be inserted. In this embodiment of the present invention the supporting means 120 may be connected to as connecting means 190 to connect the supporting means 120 to a pivot pin 170. The connecting means 190 may be connected to the supporting means 120 via any connectors such as one or more rivets 195. Other connecting means are possible such as adhesives, fasteners, screw, bolts, bonding, welding and the like or combinations thereof. In this embodiment, the connecting means 190 is housed within the distal end of the hollow housing 110 and pivots out of the aperture 140 of the hollow housing 110 upon activation and deployment and forces the distal end of the supporting means 120 into a perpendicular position to the hollow housing 110. This configuration also brings the front portion of the supporting means 120 forward to resist any inadvertent damage tissue by the distal end tip 135.

The inventive surgical device 100 may include a sheath 300 for sterilization reasons during shipping and storage. The sheath 300 may be comprised of any known material such as polymers and plastics. The sheath 300 includes a tab 310 which may be pulled so as to split the sheath 300 as the sheath 300 may include small perforations or be made of such material that the force of the pulling of the tab 310 splits the material of the sheath 300.

In use, the inventive surgical device 100 is inserted into the body cavity and placed in a location near to the tissue or organ to be retracted from the surgical site being performed on a target organ or tissue. Once inserted within the body cavity, the distal end tip 130, 135 is positioned in proximity to the non-target body organ to be retracted and contained, and the sling 400 or band 500 is deployed. Deployment is based on a mechanism 200 located opposite the distal end tip 130, 135. Deployment mechanisms 200 include but are not limited to a loop grip mechanism, pistol grip mechanism, a twist mechanism, a flexible ring member mechanism, a push/pull mechanism, or combinations thereof. The sling 400 or band 500 is moved so as to encompass the non-target tissue or organ within the sling. Thereafter the filled sling is retracted and secured within the body cavity out of the way of the instruments and devices used for the main surgery on the target body organ or tissue. At this point the surgeon use a separate instrument such as a laparoscopic grasper to further grasp and stabilize the sling 400 or band 500 and stabilize the contained retracted non-target body organ. In other circumstances the surgeon can place a temporary suture into one end of the sling 400 or band 500 to secure the sling 400 or band 500 containing the non-target body organ in a more secure retracted position within the body cavity.

When the operation is complete or when it is no longer necessary to retract the non-target body organ, the inventive surgical device 100 is moved such that the non-target body organ is released from the sling 400 or band 500. If sutures have been used to further secure the filled sling 400 or band 500, such sutures are cut. The non-target body organ is then placed to its original resting position within the body cavity. The mouth of the sling 400 or band 500 is closed via retraction of the supporting means 120 and the flexible member supporting means 120 retracted within the housing 100 or cannula of the inventive surgical device 100, with the sling 400 or band 500 partially folding within the housing 110 or cannula. The distal end of the inventive device 100 is then removed from the body cavity through the access point, such as a trocar.

In another embodiment of the present invention, a handle can be provided in connection with the inventive device 100 to facilitate manipulation thereof. Such handle may be in the shape of a loop, pistol grip and other known handles and is connected to the rod or supporting means 120.

In other embodiments of the present invention a push and pull rod mechanism 700 is employed as the activator as shown in FIG. 21. If included, the surgeon uses the loop grip 730 and moves the rod 710 in a downward movement from the loop handle 720 towards the distal end of the housing 110 towards the distal end tip 130 to activate, unfold and deploy the sling 400 or band 500 and supporting means 120 out of the aperture of the housing 110. Later, once the organ or tissue is to be released and no longer supported or retracted, the rod 710 is pulled in an upward direction so as to retract the supporting means 120 and the sling 400 or band 500 is withdrawn and pulled back into the aperture 140 of the housing 110.

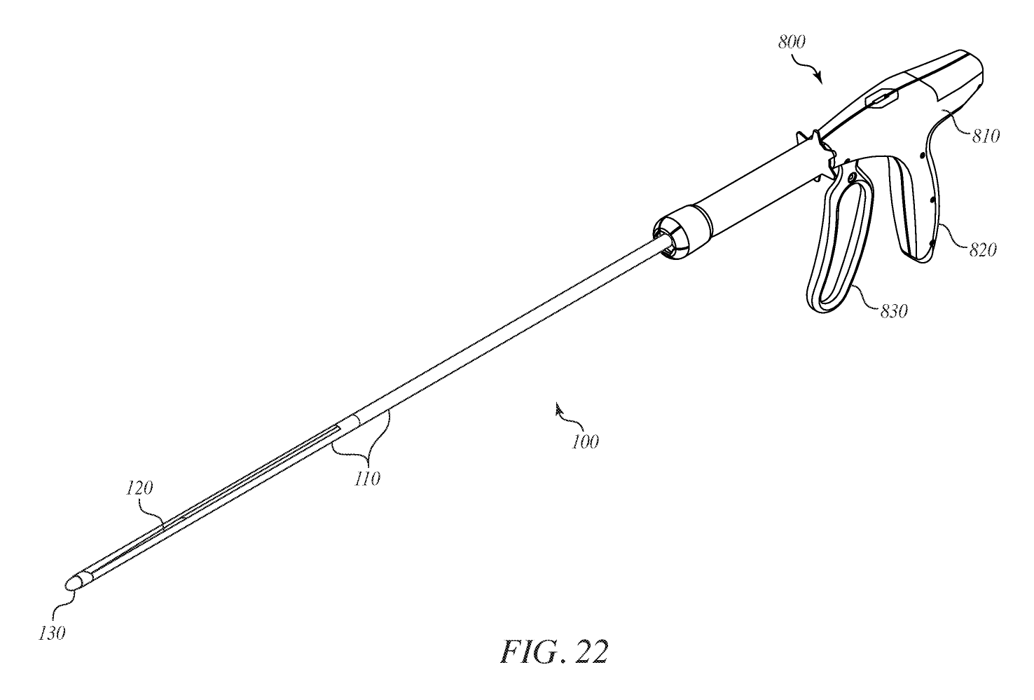

FIG. 22 shows another embodiment of the present invention including an activator or deployment means consisting of a pistol grip mechanism 800. The pistol grip deployment mechanism 800 includes a handle 810 with internal mechanisms to deploy the folded unactuated sling 400 or band 500. The handle 810 includes a trigger 830 and a proximal housing member 820. Other deployment mechanisms may be employed.

Another aspect of the present invention is a surgical kit which includes the device. The kit is stored in a sterile sealed package. The kit may include a trocar, a scissors device, a grasper device, and the sling device. The kit may optionally include a cauterizing device such as a bi-polar device. Other optional devices may be included. In one embodiment of the inventive kit all components are single use only and disposable.

Advantages of the inventive surgical support device include the configuration such that the sling or band is used to move, retract and secure a non-target body organ while a target body organ or tissue is being operated on. The contained non-target body organ has less potential for injury itself compared to use of clamps and graspers alone. Further, the surgeon has a less obstructed body cavity in which to operate once the non-target body organ or tissue is retracted and secured. There is no need for the patient to be invert into the Trendelenburg position. There is a reduced need for surgical packing (cotton, towels, sponges and the like) and the associated risk of infection and lesions and other complications with use of the inventive surgical support device. Further, the inventive surgical support device maintains a sufficient level of pneumoperitoneum during the surgical procedure. These and other problems are solved by the inventive surgical device and its methods of use. Thus, the device may reduce complications, surgical processes, time and cost.

Many possible combinations could be within the device, the methods of use, the method of treatment and the kit or system of the present invention.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

The invention has been described in terms of embodiments thereof, but is more broadly applicable as will be understood by those skilled in the art. The scope of the invention is only limited by the following claims.

We claim the following:

1. A surgical assembly for retracting and supporting an organ or tissue, comprising:
a hollow housing having an aperture on a horizontal plane;
a flexible supporting means within the hollow housing, connected on a proximal end to an activator and on a distal end to a distal end of the hollow housing;
a flexible material configured to be housed within the hollow housing until deployed by the activator to extend out of the hollow housing aperture and retract and support a body organ or tissue, connected on one side to an inner portion of the hollow housing and on the other side to the flexible supporting means;
an activator that is configured to impart a force onto the proximal end of the supporting means to urge the portion of the flexible supporting means within the hollow housing to extend out of the hollow housing aperture, to lock into an activated position, and to be released from the locked position and retracted back into the aperture of the hollow housing.

2. The surgical assembly of claim 1, wherein the flexible material is a polymer mesh material with openings sufficiently small to avoid damage to the body organ or tissue being supported, the polymer mesh material being selected from the group consisting of elastomeric material and polymeric material.

3. The surgical assembly of claim 1, wherein the flexible material is selected from the group consisting of polyurethane, polyethylene, polypropylene, vinyl, latex, polymers, plastics, and combinations thereof.

4. The surgical assembly of claim 1, wherein the flexible material is a laser cut polyester surgical mesh with a sag, the laser cut surgical mesh having a series of laser cut slots into which is slidably inserted the flexible supporting means.

5. The surgical assembly of claim 4, wherein a heat shrink forms a further connection at a location where the laser cut slots meet the flexible supporting means.

6. The surgical assembly of claim 1, wherein the flexible material is connected to an inner portion of the housing along a back portion of the aperture.

7. The surgical assembly of claim 6, further comprising a connection that connects the flexible material to the inner portion of the housing, the connection being selected from the group consisting of an adhesive, a seal, a weld and a bonding.

8. The surgical assembly of claim 1, wherein the flexible material is a polyurethane die cut film that is folded back over itself and around the flexible supporting means so that a double layer is taut to form a D shape and form a means for retracting back into the aperture of the hollow housing.

9. The surgical assembly of claim 1, wherein the flexible material has a portion wrapped around the hollow housing.

10. The surgical assembly of claim 9, further comprising a heat shrink tube over said wrapped portion of said flexible material.

11. The surgical assembly of claim 1, wherein said flexible supporting means includes inner and outer supports that are laminated together.

12. The surgical assembly of claim 1, further comprising:
a knob rotator mechanism including at least one knob with grooves, the flexible supporting means extending through the housing up to the knob rotator mechanism so that the flexible supporting means deploys by spooling around the inside of the knob and retracts by a turning in one direction versus an opposite direction of the at least one knob.

13. The surgical assembly of claim 12, wherein the at least one knob has an inner spring within that is configured and arranged to make a clicking sound in response to flexible supporting means moves between deploying and retracting.

14. The surgical assembly of claim 12, wherein the knob rotator mechanism is within the flexible supporting means and includes an end tip that houses a portion of the flexible material.

15. A method of surgical retraction or an organ or tissue, comprising the steps of:
inserting an unactivated surgical support device within an opening within human skin and into a body cavity, the surgical support device including a housing having an aperture, a flexible supporting means, a flexible material and an activator;
positioning a distal end of the surgical support device in a location near an organ or tissue to be retracted;
activating the activator and deploying the flexible supporting means and the flexible material from the aperture within the housing;
placing the deployed flexible supporting means and flexible material with force in contact with the organ or tissue so as to retract the organ or tissue;
maintaining and supporting the retracted organ and tissue for a period of time;
release force to the retracted organ or tissue and releasing to the original location within the body cavity;
deactivating the activator and withdrawing the deployed flexible supporting means and flexible material into the aperture of the housing; and
withdrawing the unactivated surgical support device from within the body cavity.

16. The method of claim 15 wherein the a flexible material is configured to be housed within the hollow housing until deployed by the activator to extend out of the hollow housing aperture and retract and support a body organ or tissue is connected on one side to an inner portion of the hollow housing and on the other side to the flexible supporting means.

17. The method of claim 15 wherein the activator is configured to impart a force onto the proximal end of the supporting means to urge the portion of the flexible supporting means within the hollow housing to extend out of the hollow housing aperture, to lock into an activated position, and to be released from the locked position and retracted back into the aperture of the hollow housing.

18. A method of assembling a surgical instrument for retracting and supporting an organ or tissue; comprising:
providing components of a surgical instrument, the components including:
a hollow housing having an aperture on a horizontal plane;
a flexible supporting means within the hollow housing;
a flexible material; and
an activator;
configuring and arranging the flexible supporting means so that the flexible supporting means is connected on a proximal end to an activator and on a distal end to a distal end of the hollow housing;
configuring and arranging the flexible material so that it is housed within the hollow housing until deployed by the activator to extend out of the hollow housing aperture and retract and support a body organ or tissue, connected on one side to an inner portion of the hollow housing and on the other side to the flexible supporting means; and
configuring and arranging the activator so that it imparts a force onto the proximal end of the supporting means to urge the portion of the flexible supporting means within the hollow housing to extend out of the hollow housing aperture, to lock into an activated position, and to be released from the locked position and retracted back into the aperture of the hollow housing.
